(19) United States
(12) Patent Application Publication
(10) Pub. No.: US 2006/0051265 A1
(43) Pub. Date: Mar. 9, 2006

(54) Apparatus and method for sorting microstructures in a fluid medium

(75) Inventors: Hisham Mohamed, Clifton Park, NY (US); Michele Caggana, Ballston Spa, NY (US); James Nathan Turner, Delmar, NY (US)

Correspondence Address: HESLIN ROTHENBERG FARLEY & MESITI PC, 5 COLUMBIA CIRCLE, ALBANY, NY, 12203, US

(73) Assignee: Health Research, Inc., Rensselaer, NY (US)

(21) Appl. No.: 11/223,432

(22) Filed: Sep. 8, 2005

Related U.S. Application Data
(60) Provisional application No. 60/607,954, filed on Sep. 8, 2004.

(51) Int. Cl. A61K 39/02

(52) U.S. Cl. 422/243

(57) ABSTRACT

An apparatus and method are provided for sorting microstructures in a fluid medium employing a receptacle having N regions of columns positioned in the receptacle between an inlet and an outlet thereof. Fluid medium introduced into the receptacle through the inlet passes sequentially through the N regions of columns before exiting through the outlet, wherein N≧2. Each region i (i=1 . . . N) of columns of the N regions of columns includes at least one row of columns spaced to define multiple fluidic channels of a respective minimum width Wᵢ. The minimum widths Wᵢ of the multiple fluidic channels of each region decrease in size in the receptacle between regions from the inlet to the outlet thereof.

### CROSS-REFERENCE TO RELATED APPLICATION

This application derives priority from U.S. Provisional Application No. 60/607,954, filed Sep. 8, 2004, entitled “Sparse Cell Isolation Device”. This Provisional Application is incorporated herein by reference in its entirety.

### STATEMENT REGARDING FEDERALLY SPONSORED RESEARCH OR DEVELOPMENT

This invention was made with support from the United States Nanobiotechnology Center and the United States National Science Foundation under Contract Nos. 0142522-04 (1999-2003) and ECS 987-6771, respectively. Accordingly, the United States government may have certain rights in the invention.

### TECHNICAL FIELD

The present invention relates in general to apparatuses and methods for sorting or fractionating microstructures such as free cells, viruses, bacteria, macromolecules, or minute particles. More particularly, the present invention relates to apparatuses and methods for sorting such microstructures in suspension in a fluid medium, including the sorting and isolation of rare microstructures within a fluid medium.

### BACKGROUND OF THE INVENTION

The sizing, separation, and study of microstructures such as free cells, viruses, bacteria, macromolecules and minute particles are important tools in many fields, including molecular biology. For example, the fractionation process, when applied to DNA molecules, is useful in the study of genes and ultimately in planning and implementation of genetic engineering processes. The fractionation of larger microstructures, such mammalian cells, promises to afford cell biologists new insights into the functioning of these basic building blocks of living creatures.

Isolating rare cells from biological fluids, including whole blood or bone marrow, is a further interesting biological application. For example, characterization of a few metastatic cells from cancer patients for further study is clearly desirable for prognosis/diagnosis. Traditional methods have not proven adequate for this particular application due to the compositional complexity of blood, with its large number of cell types.

In view of the above, further enhancements in microstructure sorting apparatuses and methods are deemed desirable.

### SUMMARY OF THE INVENTION

The shortcomings of the prior art are overcome and additional advantages are provided through the provision of an apparatus for sorting microstructures in a fluid medium. The apparatus includes a receptacle and N regions of columns positioned in the receptacle between an inlet and an outlet thereof. Fluid medium introduced into the receptacle through the inlet passes sequentially through the N regions of columns before exiting through the outlet, wherein N≧2. Each region i (i=1 . . . N) of columns of the N regions of columns includes at least one row of columns spaced to define multiple fluidic channels of a respective minimum width Wᵢ, with the minimum widths Wᵢ of the multiple fluidic channels of the at least one row of each region of columns (1 . . . N) varying between adjacent regions of columns of the N regions of columns and decreasing in size in the receptacle between regions from the inlet to the outlet thereof.

In another aspect, a method of sorting microstructures in a fluid medium is provided. The method includes: providing a receptacle having N regions of columns positioned in the receptacle between an inlet and an outlet thereof, wherein N≧2 and fluid medium introduced into the receptacle through the inlet passes sequentially through the N regions of columns before exiting through the outlet, and wherein each region i (i=1 . . . N) of columns of the N regions of columns comprises at least one row of columns spaced to define multiple fluidic channels of a respective minimum width Wᵢ, and wherein the minimum widths Wᵢ of the multiple fluidic channels of the at least one row of each region of columns (1 . . . N) vary between the N regions of columns and decrease in size in the receptacle between regions from the inlet to the outlet thereof; and employing the receptacle to sort microstructures in a fluid medium by introducing the fluid medium with the microstructures therein into the receptacle through the inlet and allowing the fluid medium to pass through the N regions of columns before exiting through the outlet, wherein differently sized microstructures separate in different regions of the receptacle dependent, in part, on physical characteristics thereof.

Additional features and advantages are realized through the techniques of the present invention. Other embodiments and aspects of the invention are described in detail herein and are considered a part of the claimed invention.

### BRIEF DESCRIPTION OF THE DRAWINGS

One or more aspects of the present invention are particularly pointed out and distinctly claimed as examples in the claims at the conclusion of the specification. The foregoing and other objects, features, and advantages of the invention are apparent from the following detailed description taken in conjunction with the accompanying drawings in which:

FIG. 1 is a partially exploded view of one embodiment of an apparatus for sorting microstructures in a fluid medium, in accordance with an aspect of the present invention;

FIG. 2 is an assembled view of the apparatus illustrated in FIG. 1, in accordance with an aspect of the present invention;

FIG. 3 is a partial view of one embodiment of an apparatus for sorting microstructures in a fluid medium, showing an interface between a first region of columns and a second region of columns, wherein the fluidic channels in the different regions of columns have different depths, in accordance with an aspect of the present invention;

FIG. 3A is a cross-sectional elevational view of the apparatus of FIG. 3 taken along line A-A, in accordance with an aspect of the present invention;

FIG. 3B is a cross-sectional elevational view of the apparatus of FIG. 3 taken along line B-B, in accordance with an aspect of the present invention;

FIG. 3C is a cross-sectional elevational view of the apparatus of FIG. 3 taken along line C-C, in accordance with an aspect of the present invention;

FIG. 4 is a plan view of one embodiment of an apparatus for sorting microstructures in a fluid medium wherein differently sized microstructures are shown isolated in different areas of the apparatus with left to right passage of fluid medium through the apparatus, in accordance with an aspect of the present invention;

FIGS. 5A-5H depict one embodiment of a process for fabricating an apparatus for sorting microstructures in a fluid medium, in accordance with an aspect of the present invention;

FIG. 6 is a plan view of another embodiment of an apparatus for sorting microstructures in a fluid medium, wherein the fluid medium is shown flowing left to right from an inlet end to an outlet end thereof, in accordance with an aspect of the present invention;

FIG. 7 is a plan view of the apparatus of FIG. 6 showing the introduction of a reverse fluid flow from the outlet end to the inlet end of the apparatus to facilitate removal of sorted microstructures from the apparatus, in accordance with an aspect of the present invention;

FIG. 8 is a plan view of an alternate embodiment of an apparatus for sorting microstructures in a fluid medium, wherein four regions of columns are illustrated, each region of columns comprising four rows of columns which define multiple fluidic channels that progressively decrease in size with each region of columns from an inlet end to an outlet end of the apparatus, in accordance with an aspect of the present invention;

FIG. 9 is a plan view of still another embodiment of an apparatus for sorting microstructures in a fluid medium, wherein axial fluid flow through the apparatus is at least one of pressure driven or via electrophoresis, and wherein cross-flow transverse to the main axial flow of fluid medium is established as well, for example by pressure or electrophoresis, in accordance with an aspect of the present invention;

FIG. 10 is a plan view of an alternate embodiment of an apparatus for sorting microstructures in a fluid medium, wherein enlarged fluidic channels of respective enlarged minimum widths are established in a first region of columns and a second region of columns by selectively omitting a column in at least some rows of columns in each region, in accordance with an aspect of the present invention;

FIG. 11 is a plan view of another embodiment of an apparatus for sorting microstructures in a fluid medium, showing an alternative arrangement of enlarged fluidic channels in respective rows in the different regions of the apparatus, in accordance with an aspect of the present invention;

FIG. 12 is a plan view of yet another embodiment of an apparatus for sorting microstructures in a fluid medium, employing selectively disposed enlarged fluidic channels in the different regions of the apparatus, in accordance with an aspect of the present invention;

FIG. 13 illustrates one embodiment of a hand-held apparatus for sorting microstructures in a fluid medium, in accordance with an aspect of the present invention; and

FIG. 14 is a plan view of an alternate embodiment of a test apparatus for sorting microstructures in a fluid medium, illustrating two separate apparatuses coupled in parallel, in accordance with an aspect of the present invention.

### BEST MODE FOR CARRYING OUT THE INVENTION

Generally stated, provided herein are apparatuses and methods for sorting microstructures such as cells, viruses, bacteria, macromolecules and minute particles (or their components) suspended in a fluid medium. The sorting apparatuses and methods disclosed herein, which are principally described below with reference to sorting or fractionation of cells in a fluid medium, separate cells based on physical characteristics and/or time to propagate through the channels of the apparatus.

In one aspect, the apparatus includes a receptacle comprising a first region of columns and a second region of columns positioned in the receptacle between an inlet and an outlet thereof. Fluid medium introduced into the receptacle through the inlet passes through the first region of columns and then the second region of columns before exiting through the outlet. The first region of columns includes at least one row of columns spaced to define multiple fluidic channels of minimum width W₁ and the second region of columns includes at least one row of columns spaced to define multiple fluidic channels of minimum width W₂, wherein W₁>W₂. Thus, in accordance with an aspect of the present invention, the minimum width Wᵢ of the multiple fluidic channels defined in each region decreases in size in the receptacle between regions from the inlet to the outlet. Numerous enhancements to this basic apparatus are described and claimed herein.

Fluid medium flow through the receptacle from the inlet to the outlet, referred to herein as “main axial flow”, can be electrophoretic, electro-osmotic, and/or pressure driven. In the structures depicted in the figures, main axial flow is assumed for purposes of example to be left-to-right. In addition to the main axial flow, a cross-flow intersecting the main axial flow, e.g., at any angle between 10 degrees and 90 degrees, can be established for a period of time, or through the entire operational period of the apparatus, or after a sample has stopped flowing through the apparatus along the main axial flow.

Defining the channels of the apparatus using discrete columns is favored over an array of continuous channels without gaps for multiple reasons. For example, having regions of discrete columns as disclosed herein: (1) allows cross-flow to be established to manipulate cells or inject a biochemical reagent in situ; (2) allows cells to migrate around a blocked area; and (3) allows cells to deform and reform as the cells traverse the apparatus, with larger, more rigid cells taking longer than smaller, more flexible cells, introducing time as an additional dimension for the sorting of cells (i.e., in addition to cell size, mobility, and mechanical properties such as rigidity).

Information from retained cells can be extracted in various ways. For example, a device can be examined (e.g., visually or through fluorescence) for presence or absence of retained cells in an expected region of the apparatus. If cells are found retained in the apparatus, then the cells can simply be counted. By way of example, chemotherapy is monitored by enumerating the number of circulating tumor cells. If the number decreases, therapy is working. If cells do not decrease in number, a different chemotherapy or an entirely different treatment can be applied. Further, cells can be lysed in situ and the released nucleic acids and proteins can be collected at the outlet. Still further, retained cells (or lysed components from cells) can be extracted intact by cross-flow through one or more cross-flow openings in the apparatus. Alternatively, main axial flow through the apparatus can be reversed, and the retained viable cells can be collected at the original inlet side of the apparatus.

Thus, cells of interest can be retained inside the device, while all other cell types migrate to the output, as the case for cancer, sperm and other applications, or cells of interest can be the cells that migrate through the entire apparatus, while other cell types become retained inside the apparatus, as would be the case for fetal cell separation. Further, surfaces of the apparatus can be treated or coated to improve fluid flow through the apparatus, or to prevent or even promote specific cell adherence within the apparatus.

As an overview, the following applications for the apparatuses and methods described herein are contemplated:
1. Isolation of fetal nucleated red blood cells from pregnant females for prenatal diagnosis and other cell studies.
2. Isolation of metastatic cells for monitoring treatment and relapse in cancer patients, and aiding in prognosis, staging, diagnosis, and treatment choices.
3. Bone marrow purging prior to transplantation.
4. Isolation of exfoliated cells from body fluids such as blood, amniotic fluid, ascites, sputum, saliva, sweat, urine, feces, cerebrospinal fluid, edema, semen, or fluid from the female genital tract.
5. Evaluation of gene expression and the metastatic process for drug development.
6. Isolating blood compartments and subpopulations therein: red blood cells, white blood cells, and platelets.
7. Isolation of cells from other biological fluids.
8. Creation of an array of single cell types for visualization.
9. Isolation of cells including bacteria, molds, and fungi from environmental samples.
10. Isolation of cells infected with a pathogen.

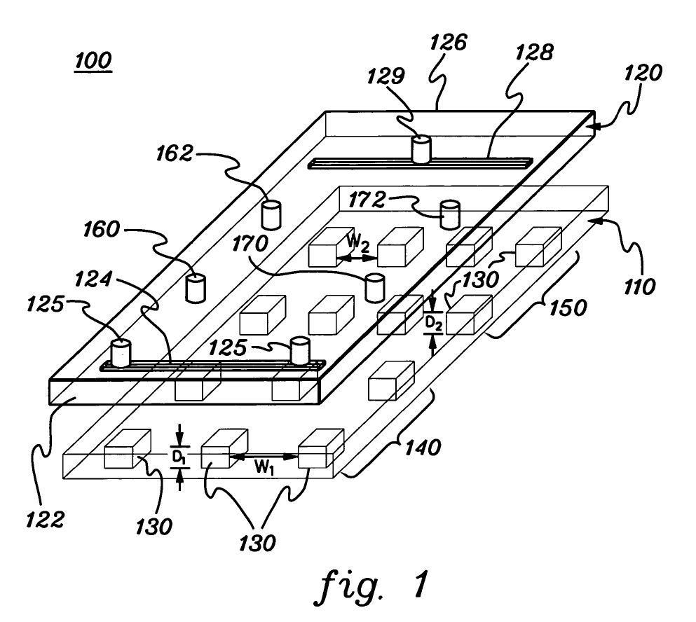

Various embodiments of the apparatuses and methods for sorting microstructures in a fluid medium in accordance with aspects of the present invention are depicted in FIGS. 1-14, and described below. In this regard, those skilled in the art will note that the figures are not drawn to scale, but rather are drawn to depict various concepts in accordance with aspects of the present invention. In actual implementation, the number of discrete columns, and therefore the number of fluidic channels defined between the columns, could be significantly greater than the numbers illustrated.

Referring to FIG. 1, one embodiment of an apparatus 100 is depicted for sorting microstructures (such as cells) in a fluid medium, in accordance with aspects of the present invention. In this figure, apparatus 100 includes a lower receptacle portion 110 and an upper receptacle portion 120, shown partially exploded. Lower receptacle portion 110 contains a plurality of columns or pillars 130 disposed in two different regions 140, 150. In region 140, columns 130 are spaced and sized to define multiple fluidic channels of minimum width W₁ and minimum depth D₁, while in region 150, columns 130 are spaced and sized to define multiple fluidic channels of minimum width W₂ and minimum depth D₂, wherein W₁>W₂.

Upper receptacle portion 120 of apparatus 100 is sized and configured to mate with lower receptacle portion 110, and includes a first, inlet end 122 having an inlet plenum 124 provided therein and a second, outlet end 126, having an outlet plenum 128 provided. Inlet plenum 124 is fed via one or more inlets 125 through which fluid medium with microstructures disposed therein is introduced into the apparatus. Outlet plenum 128 is in fluid communication with at least one outlet 129 through which fluid medium and any microstructures passing through the apparatus are withdrawn. Upper receptacle portion 120 further includes, in this embodiment, multiple inlet cross-flow openings 160, 162 and multiple outlet cross-flow openings 170, 172. Openings 160, 162, 170 & 172 facilitate the introduction and removal of a cross-flow fluid through the apparatus. For example, these openings may be aligned to facilitate the removal of microstructures sorted into the different regions, or the introduction of a biochemical agent during passage of fluid medium through the apparatus from the inlet plenum to the outlet plenum. If desired, cross-flow openings (as well as main inlets and outlets) could be provided in lower receptacle portion 110 as well.

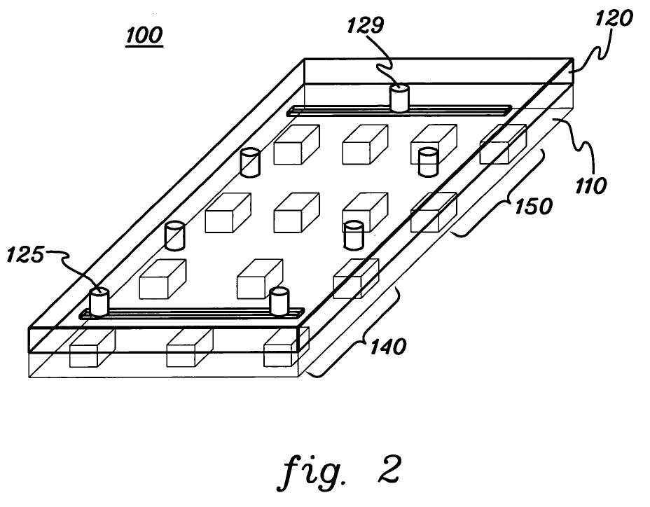

FIG. 2 is an assembled embodiment of apparatus 100, wherein fluid medium introduced into the receptacle (defined by lower receptacle portion 110 and upper receptacle portion 120) through inlet 125 passes sequentially through region 140 of columns and then region 150 of columns before exiting through outlet 129. Although shown as comprising two rows of columns, each region 140 & 150 comprises at a minimum at least one row of columns spaced to define multiple fluidic channels of the desired, respective minimum width Wᵢ, wherein minimum width Wᵢ decreases in size from the first region of columns closest to the inlet, to the last region of columns closest to the outlet. Further, any number N of regions of columns can be employed within the receptacle, with only two regions of columns being illustrated in FIGS. 1 & 2 for simplicity.

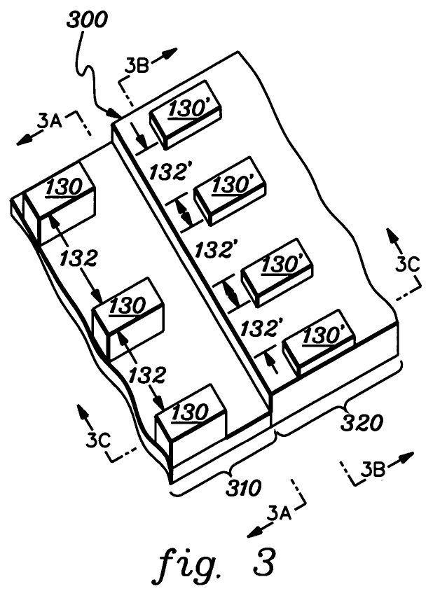

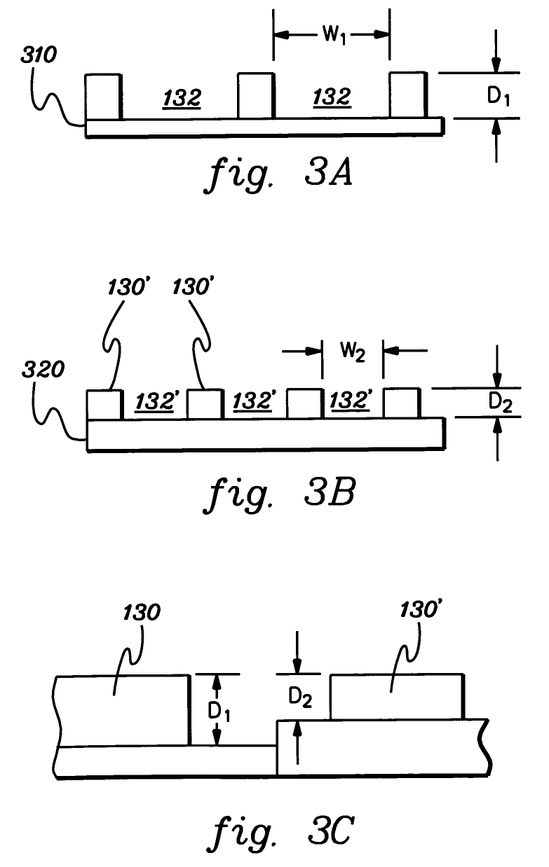

FIGS. 3-3C depict a partial embodiment of a lower receptacle portion 300 of another apparatus in accordance with an aspect of the present invention. As shown, receptacle portion 300 includes a first region 310 of columns and a second region 320 of columns. First region 310 has at least one row of columns 130 spaced to define multiple fluidic channels 132 of minimum width W₁, while second region 320 has at least one row of columns 130′ spaced to define multiple fluidic channels 132′ of minimum width W₂. As shown in the cross-sectional views of FIGS. 3A & 3B, the multiple fluidic channels 132 defined by spaced columns 130 also have a minimum depth D₁, and the multiple fluidic channels 132′ defined by spaced columns 130′ have a minimum depth D₂, wherein D₁>D₂. FIG. 3C further illustrates this difference in minimum depth between regions of columns of the apparatus. By providing a larger minimum depth D₁ in region 310, larger microstructures are allowed to accumulate within the region without blocking flow of medium and smaller sized microstructures in the fluid medium. The width and depth of the channels in the various regions of the apparatus are characterized herein as comprising “minimum width” and “minimum depth”, respectively. These terms imply that the width of the defined fluidic channels can vary within a region dependent (for example) on the cross-sectional configuration of the columns. By way of example, circular columns define fluidic channels of varying width, while square or rectangular columns define fluidic channels of substantially uniform width. Still further, the fluidic channel width may comprise a minimum width at an inlet of the channel, and then broaden to a larger width at an outlet thereof. Thus, various configurations are encompassed by the present invention. Still further, the depth of the fluidic channels in different regions of the apparatus may be the same or different, depending on the particular application.

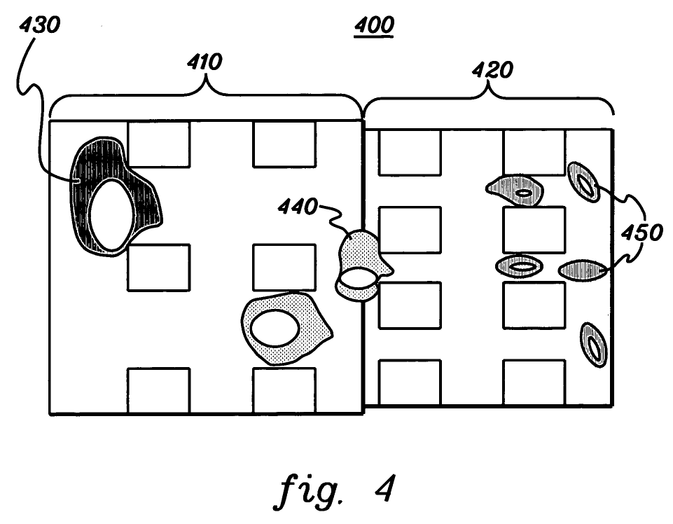

FIG. 4 is a plan view of one embodiment of an apparatus 400, in accordance with an aspect of the present invention. Apparatus 400 comprises a receptacle which has a first region 410 of columns and a second region 420 of columns. Again, each region has at least one row of columns, with two rows being shown for each region in this example. The columns are spaced and sized to define multiple fluidic channels of respective minimum widths Wᵢ. In operation, larger cells 430 are blocked at the first region of columns, medium sized cells 440 pass through the first region and are blocked at the second region of columns, and smaller cells 450 migrate through both the first region and the second region, thereby achieving sorting of cells 430, 440 & 450.

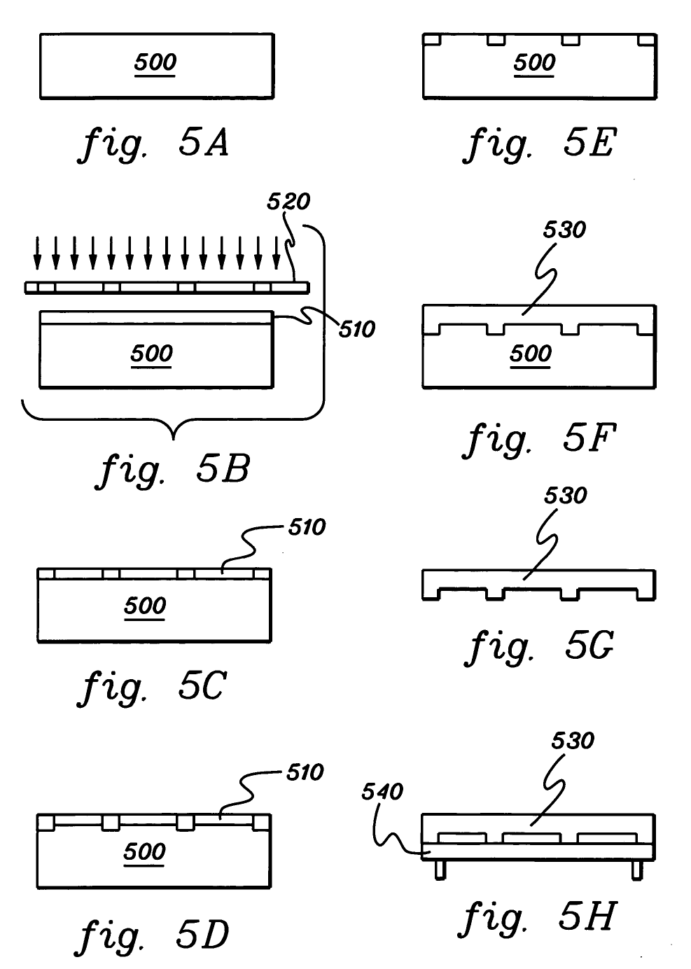

One approach for fabricating an apparatus in accordance with the present invention is depicted in FIGS. 5A-5H. Initially, a complementary image of the apparatus' channel design is laid out on a design tool, then transferred to a pattern generator to make a mask. The complementary image is then used instead of the original design since a wafer is to be employed as a master to make device molds as described below. Wafer 500 (FIG. 5A) is patterned employing a mask and standard photolithography techniques (FIG. 5B). That is, a photoresist 510 is spun onto wafer 500, which is then exposed through mask 520 using a UV source. This is followed by wafer development in a solvent to remove the exposed photoresist, with the resultant structures shown in FIG. 5C. Next, channels are etched into wafer 500 using, for example, deep reactive ion etching (FIG. 5D). The wafers are then plasma-cleaned to remove the photoresist (FIG. 5E). The silicon wafers can then be soaked in a 2% Aquaphobe™ (marketed by Gelest, Inc., Morrisville, Pa.) in dry hexane for two minutes, followed by a 24 hour drying at room temperature (or 20 minutes at 115° C.) to render the wafers hydrophobic. A mirror image of the apparatus is molded using an elastomer/polymer 530 (FIG. 5F) (which may be a transparent elastomer/polymer). This elastomer/polymer can be mixed with a curing agent, then poured onto the silicon wafer and de-gassed to remove air bubbles. The elastomer/polymer is cured at high temperature and separated from the silicon wafer to create a mirror replica of the structure on the silicon wafer (FIG. 5G). This is the lower receptacle portion of the apparatus (shown inverted in the figure). The upper receptacle portion is similarly molded, but without the channels. For example, the elastomer/polymer can be used to create a mold of a regular glass cover slip. This mold is then used to make the featureless upper receptacle portion. The upper receptacle portion is then machined to create the desired holes and plenums/reservoirs. (Alternatively, the holes and plenums/reservoirs could be molded into the upper receptacle portion.) Both the upper receptacle portion and the lower receptacle portion are then plasma-cleaned to promote adhesion and to make the surface hydrophilic to enhance flow. The two portions are finally pressed together to seal the apparatus and form the functional device (FIG. 5H).

Those skilled in the art will note that there are many variations to the above-outlined fabrication protocol. Different polymers will have different process conditions, and certain polymers cannot be used directly on, for example, silicon wafers, so that an intermediate molding step may be required. In such a case, the silicon wafer might have the exact device structure, not the complementary image, since two molding steps will take place.

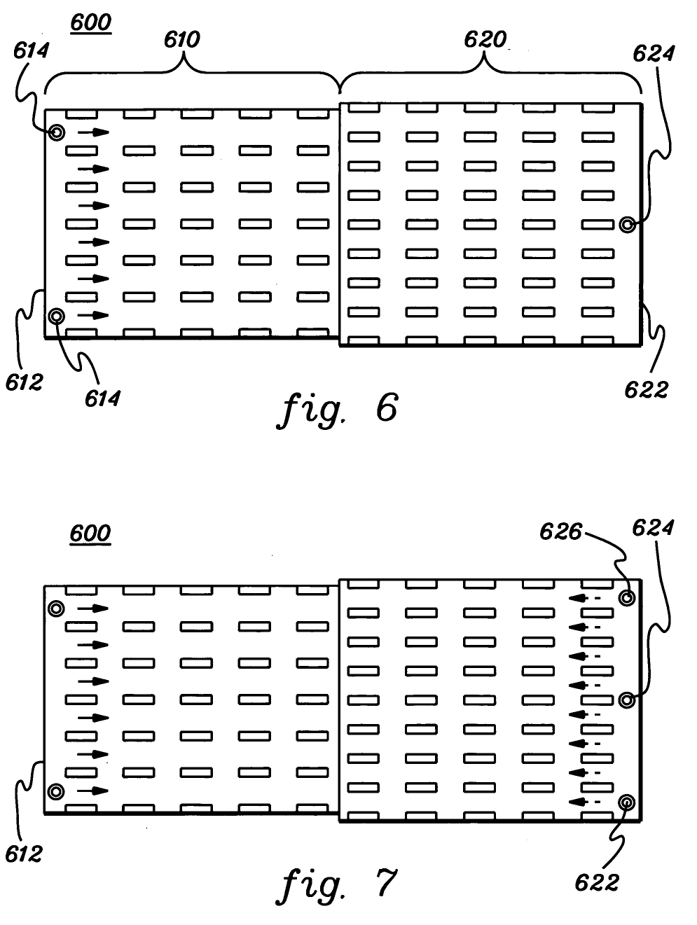

FIGS. 6 & 7 depict further embodiments of an apparatus 600 in accordance with an aspect of the present invention. As shown, apparatus 600 includes a first region 610 of columns and a second region 620 of columns. Microstructures and fluid medium flow from an inlet end 612 to an outlet end 622. The microstructures and fluid medium are introduced through one or more inlets 614 at inlet end 612 of the apparatus and are removed via one or more outlets 624 at outlet end 622 of the apparatus. Further, region 610 of columns is shown to comprise five rows of columns, and region 620 five rows of columns. Again, within a row, the columns are spaced to define the multiple fluidic channels (generally oriented with the main axial flow), which are larger in the first region of columns than the second region of columns. During operation, various techniques can be employed to extract retained cells from apparatus 600. For example, as shown in FIG. 7, flow through the apparatus can be reversed, with any retained cells being collected at the original input end 612. With this extraction approach, additional outlets 626 may be employed either in fluid communication with original outlet 624 via a plenum, or separate.

As a more specific example, intact cancer cells can be removed from the apparatus by reversing the fluid medium flow after cancer cells have been retained and all blood cells have been flushed through the apparatus. In order to ensure that only retained cancer cells are extracted, the inlet tubing can be changed after cell loading, but prior to cell reversal (or a different inlet could be used). Additionally, the apparatus can be flushed for a period of time (for example, 20 minutes) using medium only, prior to flow reversal. Reversing the flow, while keeping both ends of the apparatus wet at all times, can be achieved by filling the output plenum with fluid medium one or two seconds prior to reversing the flow. In cell lysis, removal of cell lysed components from captured cells can be accomplished by flowing water or a buffer through the apparatus, with the desired component being collected via the output plenum.

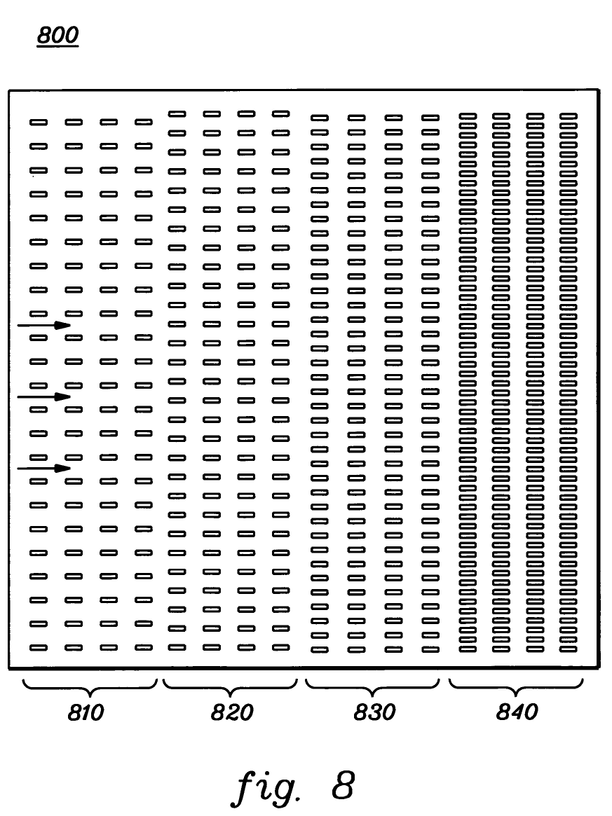

FIG. 8 is a plan view of still another embodiment of an apparatus 800 for sorting microstructures in a fluid medium, in accordance with an aspect of the present invention. In this embodiment, four regions, 810, 820, 830 & 840 are shown, with each region comprising four separate rows of columns. The columns within each row are spaced to define the multiple fluidic channels of the respective minimum widths Wᵢ (wherein i=1 . . . 4). Assuming that the main axial fluid flow is left-to-right, then the minimum width Wᵢ of each region progressively decreases from region 810 through regions 820 & 830 to region 840 as shown. Similarly, if desirable for a particular application, the columns of the different regions can be sized so that the depths within each region also vary from inlet end to outlet end of apparatus 800. Further, depending on the application, the number of regions of columns, as well as the number of rows within each column can vary. Again, in this regard, the embodiments presented herein are provided by way of example only. As a specific example, channel widths within the regions may respectively be 20, 15, 10 and 5 μm wide, and 20, 15, 10 and 5 μm deep. Further, each region may contain 1,500-2,000 channels arranged in multiple parallel rows. In one embodiment, each region is 1.5 cm long and 3 cm wide, resulting in a 6 cm long by 3 cm long apparatus.

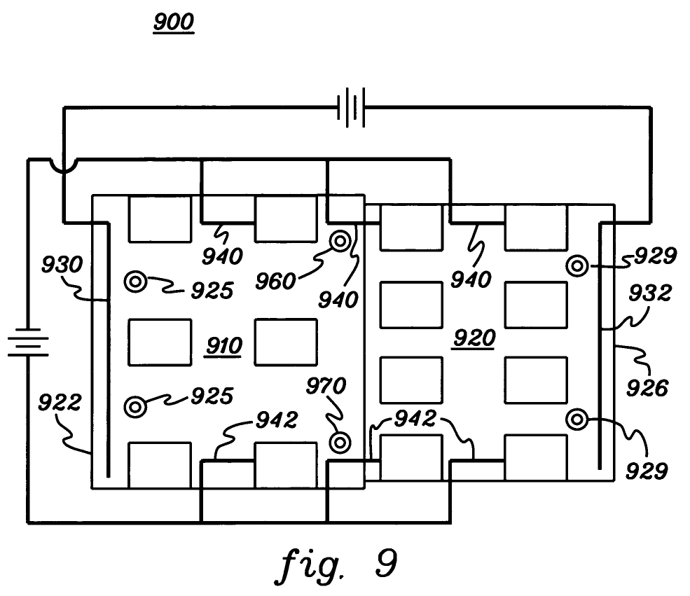

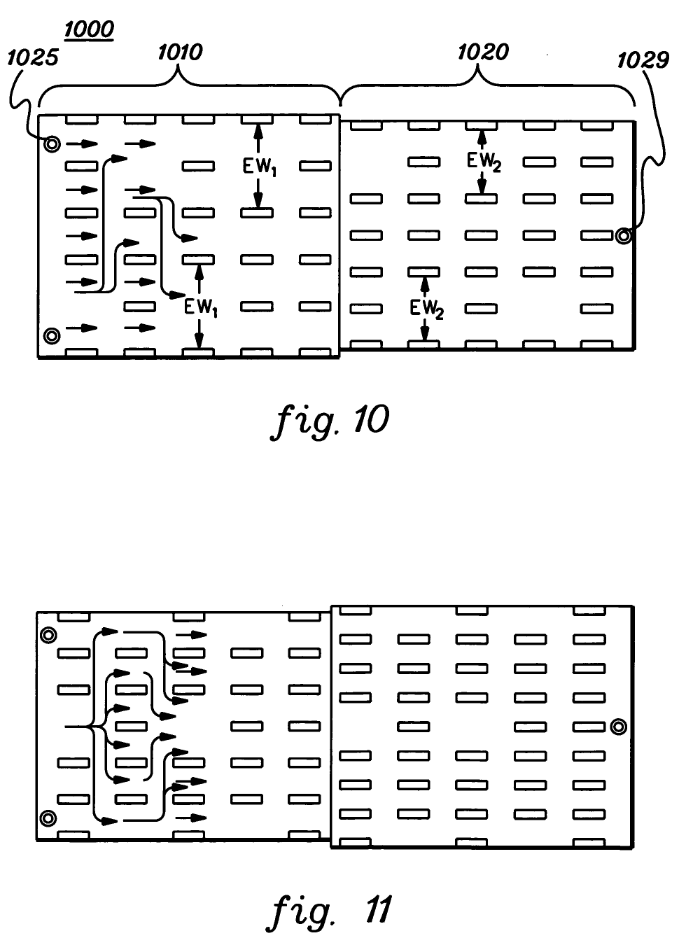

In the apparatus examples described above, it is assumed that fluid medium flows through the apparatus via a pressure differential between the inlet and outlet, for example, through the application of a vacuum pressure at the outlet of the apparatus. FIG. 9 depicts a further embodiment of an apparatus 900 in accordance with an aspect of the present invention. This sorting apparatus 900 again includes: a first region 910 of columns and a second region 920 of columns, wherein multiple fluidic channels are defined between rows of columns in each region, and the minimum width of the channels varies from the first region to the second region; and fluid medium is introduced through inlets 925 at an inlet end 922, and withdrawn through outlets 929 at an outlet end 926. In this embodiment, however, electrodes 930 and 932 are added at inlet end 922 and outlet end 926, respectively, of the apparatus. These electrodes drive axial electrophoretic flow of the fluid medium, either as an alternative to or in combination with, pressure-driven flow (e.g., by the application of a vacuum to outlets 929).

Electrodes 940 & 942 can also be added to the sides of apparatus 900 to facilitate movement of microstructures in a direction other than the main axial flow direction. For example, a cross-flow fluid can be introduced through a side inlet side 960 and removed through a side outlet 970, with cross-electrophoresis employed in extracting isolated cells at the interface between first region 910 and second region 920. Again, the use of electrode driven cross-flow could be an alternative to a pressure driven cross-flow, or in combination therewith.

Electrodes can be added to the sorting apparatus by inserting thin platinum wires during a final molding step. These wires could be externally mounted onto the apparatus, with a simple fixture used to position the electrodes, or they could be deposited in the apparatus using additional mask and photolithography steps to transfer the electrode pattern into the apparatus and then deposit the electrodes.

FIGS. 10-12 depict further embodiments of an apparatus in accordance with aspects of the present invention. In these embodiments, the apparatus is modified to include within each region of columns, one or more rows of columns which have at least one enlarged fluidic channel of minimum width EWᵢ. These enlarged fluidic channels in the one or more rows of each region facilitate cross-movement of fluid medium within the apparatus and inhibit larger microstructures from blocking flow of smaller microstructures through the apparatus.

In the embodiment of FIG. 10, apparatus 1000 again includes a first region 1010 of columns and a second region 1020 of columns, with each region of columns containing five rows of columns. The columns in region 1010 are spaced to define multiple fluidic channels of minimum width W₁, while those in region 1020 are spaced to define multiple fluidic channels of minimum width W₂, wherein W₁>W₂, so that the widths of fluidic channels decrease in size in the receptacle between regions from the inlet to the outlet of the apparatus. Fluid medium introduced through one or more inlets 1025 flows through the regions of the receptacle, and is then removed through one or more outlets 1029. As shown, the first four rows in region 1010 each have at least one enlarged fluidic channel of minimum width EW₁ defined by selectively removing (i.e., not defining) a column in each of these rows. The last row in region 1010 at the interface with region 1020 contains a full complement of columns to ensure that larger microstructures are retained in region 1010. Similarly, the first four rows of region 1020 each have at least one enlarged fluidic channel of minimum width EW₂, again, to facilitate movement of the fluid medium through the apparatus and the prevention of medium sized cells from blocking smaller sized cells from migrating through the second region. Additionally, in the embodiment of FIG. 10, the enlarged fluidic channels in first region 1010 and second region 1020 are unaligned in successive rows, being alternatively disposed along opposite sides of the apparatus. This positioning of the enlarged openings facilitates cross-movement of fluid medium within each region, thereby ensuring better distribution of the fluid medium and better microstructure flow within the apparatus.

FIG. 11 depicts an alternative apparatus embodiment wherein the enlarged fluidic channels alternate in successive rows between a center of the receptacle and a side edge of the receptacle, again, to promote cross-movement of fluid medium within each region of the receptacle for better distribution and flow.

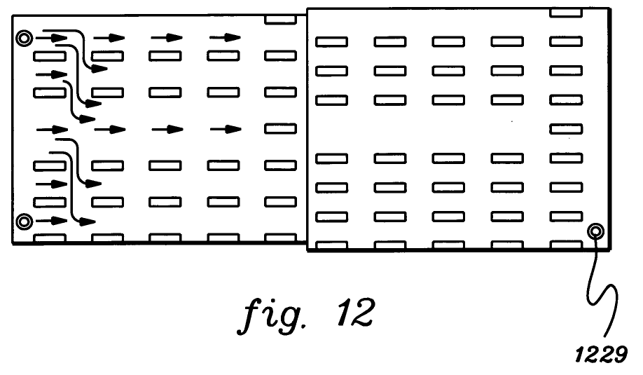

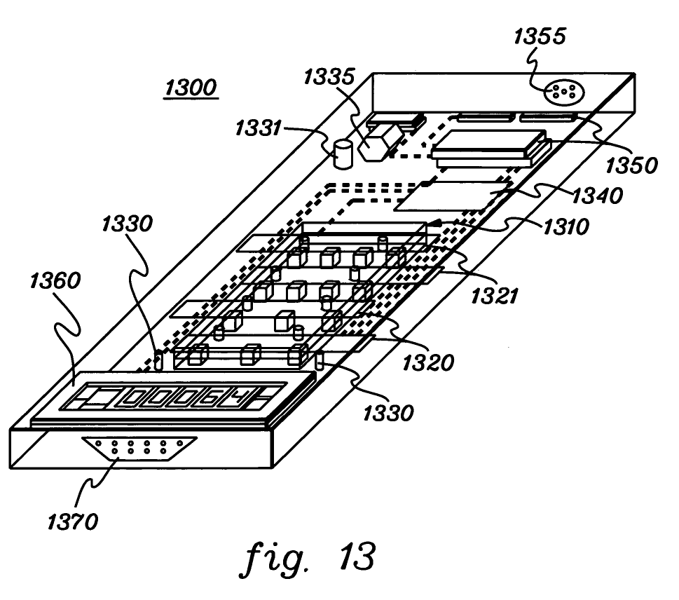

In FIG. 12, an apparatus embodiment is depicted wherein the enlarged fluidic channels in each region of columns are disposed at a center and one side of the region (again, with the exception of the last row of columns in each region, which has a full complement of columns to ensure that no larger microstructures than appropriate escape the respective region). In this embodiment, outlet 1229 for the apparatus is disposed at one side of the outlet end of the receptacle. This one side is opposite to that having the aligned enlarged fluidic channels. This positioning promotes cross-movement of microstructures and fluid medium within the apparatus as the fluid medium is driven to outlet 1229, thereby ensuring better distribution and flow.

Those skilled in the art will note from the embodiments of FIGS. 10-12 that the enlarged fluidic channels can be disposed anywhere within the different regions of the apparatus to promote main axial flow and/or cross-movement of microstructures and fluid medium.

FIG. 13 illustrates one embodiment of a hand-held device 1300 for sorting microstructures in accordance with an aspect of the present invention. Device 1300 includes an apparatus 1310 for separating and isolating microstructures as described above in connection with the embodiments of FIGS. 1-12. As in the above embodiments, the apparatus is characterized by having N regions of columns (N≧2), wherein at least one row of columns in each region is spaced to define multiple fluidic channels of a respective minimum width Wᵢ, and wherein the minimum widths Wᵢ of the multiple fluidic channels decrease in size in the receptacle between regions from the inlet to the outlet thereof. Apparatus 1310 is preferably configured within the device to be a replaceable/disposable cartridge.

In the embodiment of FIG. 13, device 1300 includes, for example, multiple sets 1320, 1321 of aligned light source/detectors disposed around a transparent apparatus 1310. For example, within each set, an LED or photodiode array could be disposed above the receptacle and detectors below the receptacle to detect retained cells within the receptacle. These sets of cell sensors could be employed at various locations about the receptacle 1310. Input reservoirs 1330 facilitate loading of a sample and reagents into the apparatus, and can also be employed as collection reservoirs for retained cells within the receptacle subsequent to flow reversal through the receptacle. An output collection reservoir 1331 is coupled via a fluidic line (not shown) to the outlets of the receptacle and a pump 1335 facilitates flow of fluid medium through the receptacle. An electronic controller 1340 is provided for detection and read-out of cells. Batteries and power supply 1350 power the hand-held device. A power port 1355 may be employed to recharge the batteries. A read-out and control display 1360 is provided at one end of the device, as well as a data port 1370 to program or download readings from the device.

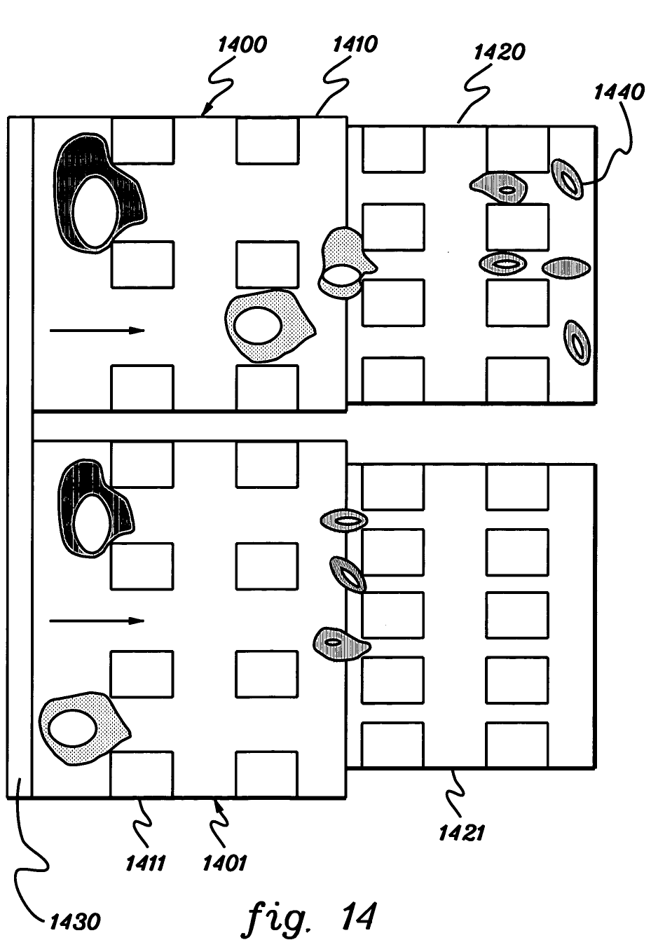

If all regions cannot practically be accommodated in one receptacle, then it is possible to group two or more receptacles in parallel, as depicted in FIG. 14. In this case, two receptacles 1400, 1401 are shown. Receptacle 1400 includes a first region 1410 and a second region 1420, while receptacle 1401 includes a first region 1411 and a second region 1421. The rows of columns in each region 1410, 1411, 1420, 1421 are spaced to define multiple fluidic channels of a respective minimum width Wᵢ. That is, the width between columns in each region is different from the width between columns in all other regions. In this example, four different channel widths are thus accomplished employing two receptacles in parallel. Each receptacle is coupled to an inlet reservoir or plenum 1430 which distributes the fluid medium with the microstructures to be sorted into the receptacles. The apparatus is particularly advantageous for use in testing for critical dimensions of cells of interest. In this example, cells 1440 were not retained by receptacle 1400, but were retained by receptacle 1401 at the interface between regions 1411 & 1421. The transition from one region to the next is in small increments to accurately identify critical dimensions of a cell and the channel widths that will retain the types of cell of interest.

Those skilled in the art will note from the above discussion that an apparatus in accordance with aspects of the present invention requires no prior information about a cell's surface marker or genetic abnormality. The apparatus can retrieve live cells for further analysis, where many existing apparatuses cannot. Further, the apparatus described is less laborious and expensive than existing approaches and can be widely implemented. For example, an apparatus such as depicted in FIG. 14 can be employed as a test apparatus with many more regions and rows of columns than depicted.

To summarize, a new technology has been develop to isolate cells, e.g., one or two or more per milliliter of volume, from biological fluids such as blood, bone marrow, amniotic fluid, ascites, sputum, sweat, urine, feces, cerebrospinal fluid, edema, semen and fluid from the female genital tract. The apparatus presented has applications in a wide variety of areas where there is a need to isolate, remove, manipulate and/or monitor individual cells. The isolation of cells is based on their physical properties requiring no prior knowledge about surface markers or gene expression. By implementing specific versions of the device, the application areas become many fold and examples include: non-invasive prenatal diagnostic testing; cancer detection; bone marrow purging; and other applications involving the measurement, sorting or removal of individual biological cells. The apparatus is simple to use and includes a design for clinical testing applications using standardized testing protocols. For testing applications, cells can be isolated in less than one hour with only one sample manipulation step prior to apparatus loading (i.e., dilution of fractionation). No operator time is required during a test and the receptacle or cartridge is disposable.

The apparatus fabrication process described herein allows for high volume production at low cost, permitting the device to be disposable, and thereby eliminating contamination concerns. In addition, the flexibility of the technology platform lends itself to custom cartridge type applications for specific solution concerns. The flexible device design and its micro-scale size also lend it to being interfaced with external devices for additional downstream applications. For example, the modular design allows it to be connected to existing genetic testing devices once cells of interest are isolated.

The following examples are based on cartridges specific to various applications.

### EXAMPLE 1

### Non Invasive Prenatal Genetic Testing

For this application, maternal blood is drawn intravenously and the apparatus is used to isolate the fetal nucleated red blood cells (fNRBCs), thus eliminating the current invasive procedures such as amniocentesis or chorionic villus sampling. The test can be performed starting from the sixth week of gestational age, and has the potential to help support a clinical diagnosis or screen for a particular health problem throughout the duration of the pregnancy. Theoretically, testing would not be limited to certain gestational periods. In addition, the technology is likely to reduce the cost associated with the prenatal diagnostic testing.

### EXAMPLE 2

### Metastatic Cell Isolation

In this application, the apparatus is implemented to isolate metastatic cells from a cancer patient's blood to monitor treatment and detect relapse at an earlier point than is currently possible. Blood can be sampled more frequently, leading to more detailed follow-up and improved patient management, including “personalized” drug regimens. Further research may confirm that testing can be performed on cells isolated from the peripheral circulation, instead of using more invasive procedures such as needle aspiration and testing of non-metastatic cells. Additionally, isolating metastatic cells may provide a means to study cell tumor gene expression in order to identify efficacious drugs at the time of diagnosis and to aid in prognosis and staging.

### EXAMPLE 3

### Bone Marrow Purging

Prior to transplantation, the apparatus can be used to “filter” cancer cells from bone marrow. Passing the sample through the device retains the cancerous cells while allowing the healthy cells to migrate through where they are collected at the opposite end. This cancer free sample can then be transplanted back into the patient.

Experiments:

First-generation devices had channels that were 5, 10, 15 or 20 μm wide, and 5, 10, 15 or 20 μm deep. The channel width and depth were constant across the entire device. Experimentations with various combinations of channel widths and depths were performed. When the channel dimentions were larger than the neuroblastoma (NB) cell (≈10 μm in diameter), such cells passed through the entire device without resistance. (The used NB cell line was: SK-N-MC, and was purchased from ATCC, Manassas, Va.) A reproducible flow pattern could not be established when the channel dimensions are comparable to or smaller than the cellular dimensions; that is, cells adhered at times, particularly when a concentrated pure population was loaded, to the column walls at the entrance of the channel, due to high concentration, slow flow, and wall roughness reproduced from the etch process.

Second-generation devices were designed by integrating all four channel segments. Channels narrowed through the device with widths of 20 μm, then 15 μm, then 10 μm, and finally 5 μm wide. Channel depths, constant within a given apparatus, were 20 μm, 15 μm, 10 μm, or 5 μm. Each segment was composed of ˜1,800 channels of the same dimensions arranged in ˜375 parallel arrays. The first two regions were designed to ensure an even flow distributed over the entire width of the apparatus, while the two subsequent smaller regions were designed to optimize separation. The goal was to use the device to model separation of NB cells from human whole blood. In order to achieve this goal, the “critical dimensions” for blood were established first to better characterize the apparatus, and then, to extend its use to additional cell fractionation applications beyond the isolation of NB cells.

The output connector was connected to house vacuum (≈23 in Hg, +/−10% maximum variation) for each experiment. A wetting solution, 2% tetra (ethylene glycol)-dimethyl-ether solution (Sigma-Aldrich) in Eagle's minimal essential medium (EMEM), was used to enhance cell migration through the device. This solution was tested for its effects on cell viability, and was found to have little effect for short-term exposure; since cell are loaded in straight medium after wetting, and flow is established. The wetting and test solutions were pressure-driven, from the inlet reservoir to the outlet reservoir (negative pressure being applied to the output side). Once the complete apparatus was wet, the cell solution was introduced. All experiments were completed under a fluorescence microscope equipped with a digital video camera.

Experiments with human whole blood proved that the second generation of devices had gaps and depths larger than required for peripheral blood fractionation; as a result, all cells from healthy human whole blood crossed the entire apparatus without resistance.

When cultured NB cells were tested with the 5 μm deep device, cells were retained consistently in the first few rows of the 10 μm channels. NB cells were cultured in EMEM with 10% fetal bovine serum (FBS), 5% L-Glutamine, and 5% PENSTREP, at 37° C. and under 5% CO₂. Cultured NB cells were first tested in medium only. A challenge with these cells was adhesion to the channel walls, particularly in earlier segments, upstream of where freely moving cells were retarded based on the limitations of channel size. The roughness of the column wall from the Bosch etching process, reproduced with high fidelity in the polyurethane, and the slow flow at the beginning of the device due to the device length and depth, contributed to the problem. A shorter device (3.5 cm), with deeper channels (20 μm), reduced the resistance to flow and alleviated the adhesion problem.

Based on the results from the second-generation device, a third-generation device with channel spacing at 15 μm, 10 μm, 5 μm, and 2.5 μm intervals was fabricated as described above, for additional experimentation with human blood. All channels were 5 μm deep. Either whole blood diluted in medium (1:10 v/v; blood was obtained from a healthy adult volunteer in our laboratory), or isolated mononuclear cells were used in the device. Mononuclear cells were isolated by 1:1 dilution (v/v) with phosphate buffered saline (PBS, pH 7.4) and centrifugation on a density gradient (Ficoll-Paque). After Ficoll separation, the mononuclear cell layer contains adult red blood cells at very low concentrations. When the mononuclear cell layer was used in the 5 μm deep devices, the white cells were retained at the beginning of the 2.5 μm wide channel segment, while red blood cells, identified by morphology under the microscope, traversed the entire device. The previous experiment was repeated, and the blood was stained with nucleic acid stain (SYTO Red, Molecular Probes, Inc., Eugene, Oreg.) to differentiate nucleated white blood cells from enucleated mature red blood cells. These results using fluorescence confirmed our bright field microscope results, the retained cells were indeed nucleated. Isolated red blood cells, when present in very low concentration in medium as described above, traversed the entire apparatus without resistance down to the 2.5 μm channel width. Here, they slowed down but still passed through to the output reservoir. Adult red blood cells in higher concentration, 1:100 v/v in medium encountered a higher resistance at the 2.5 μm wide channels and slowed down to the point that they aggregated at the channel exits, and were retained. Higher concentrations of adult red blood cells led to uncontrolled cell aggregation, even in upstream segments wider than the 2.5 μm channels.

Adult whole human blood diluted in medium (EMEM), at 1:10 v/v, and spiked with cultured NB cells, was tested in the apparatus. As expected, the NB cells were retained at the beginning of the 10 μm wide by 20 μm deep channels, while adult blood cells passed through the entire device without resistance, proving our hypothesis and duplicating the results obtained by testing the cell populations separately.

To summarize, the isolation of cultured NB cells has been demonstrated when mixed with whole blood, based solely on size and deformation characteristics. With the exception of dilution of the blood sample with medium, no other sample manipulation was required. Using the second-generation device, NB cells were consistently isolated in the 10 μm wide by 20 μm deep channels, while blood cells migrated to the output reservoir. Experiments using 2 mL of whole blood, before dilution, took approximately 2-3 hours. This method thus presents great advantages, in both cost and time, over existing methods such as FACS and MACS, which require 2 days to allow for manual manipulation, testing, and result assessment. The time frame can be reduced yet further by optimizing the microfluidics and/or increasing the width, by making the device even more massively parallel, and by decreasing the length. This device will be used to capture metastatic cells in the patient's peripheral circulation, for later characterization by molecular means. Additionally, this device will be used to capture tumor cells in bone marrow for molecular analysis and/or for purging.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. An apparatus for sorting microstructures in a fluid medium, said apparatus comprising:
a receptacle;
N regions of columns positioned in the receptacle between an inlet and an outlet thereof, wherein fluid medium introduced into the receptacle through the inlet passes sequentially through the N regions of columns before exiting through the outlet, wherein N≧2; and
wherein each region i (i=1 . . . N) of columns of the N regions of columns comprises at least one row of columns spaced to define multiple fluidic channels of a respective minimum width Wᵢ, and wherein the minimum widths Wᵢ of the multiple fluidic channels of the at least one row of each region of columns (i . . . N) decrease in size in the receptacle between regions of columns of the N regions of columns from the inlet to the outlet thereof.

2. The apparatus of claim 1, wherein the N regions of columns comprise a first region of columns and a second region of columns positioned in the receptacle between the inlet and the outlet thereof, wherein fluid medium introduced into the receptacle through the inlet passes through the first region of columns and then the second region of columns before exiting through the outlet, and wherein the first region of columns comprises at least one row of columns spaced to defined multiple fluidic channels of minimum width W₁ and the second region of columns comprises at least one row of columns spaced to define multiple fluidic channels of minimum width W₂, wherein W₁>W₂.

3. The apparatus of claim 2, wherein the at least one row of columns of the first region of columns are sized to define multiple fluidic channels of minimum depth D₁ and the at least one row of columns of the second region of columns are sized to define multiple fluidic channels of minimum depth D₂, wherein D₁≠D₂.

4. The apparatus of claim 3, wherein minimum depth D₁ is greater than minimum depth D₂.

5. The apparatus of claim 2, wherein the first region of columns comprises multiple rows of columns, each row having columns spaced to define multiple fluidic channels of minimum width W₁, and wherein the second region of columns comprises multiple rows of columns, each row having columns spaced to define multiple fluidic channels of minimum width W₂.

6. The apparatus of claim 5, wherein at least some rows of the multiple rows of the first region of columns each have at least one enlarged fluidic channel of minimum width EW₁ and wherein at least some rows of the multiple rows of the second region of columns each have at least one enlarged fluidic channel of minimum width EW₂, wherein EW₁>W₁ and EW₂>W₂.

7. The apparatus of claim 6, wherein the enlarged fluidic channels of minimum width EW₁ of the first region of columns are unaligned in successive rows of the multiple rows of columns of the first region of columns, and wherein the enlarged fluidic channels of minimum width EW₂ of the second region of columns are unaligned in successive rows of the multiple rows of columns of the second region of columns.

8. The apparatus of claim 7, wherein the enlarged fluidic channels of minimum width EW₁ and EW₂ are disposed in the first and second regions of columns, respectively, to facilitate non-axial movement of fluid medium within the receptacle in addition to axial flow of fluid medium through the receptacle from the inlet to the outlet thereof.

9. The apparatus of claim 8, wherein the enlarged fluidic channels of minimum width EW₁ and EW₂ are further disposed within the receptacle to facilitate flow of fluid medium through the receptacle from the inlet to the outlet thereof, and wherein the first region of columns comprises at least one row of columns spaced to define only multiple fluidic channels of minimum width W₁, and wherein the second region of columns comprises at least one row of columns spaced to define only multiple fluidic channels of minimum W₂.

10. The apparatus of claim 6, wherein the outlet is disposed at an outlet end of the receptacle, and wherein the inlet is disposed at an inlet end of the receptacle, and wherein the outlet is disposed at one side of the outlet end of the receptacle, and the enlarged fluidic channels of minimum width EW₁ of the first region of columns are aligned in at least some successive rows of the multiple rows of columns of the first region of columns, and the enlarged fluidic channels of minimum width EW₂ of the second region of columns are aligned in at least some successive rows of the multiple rows of columns of the second region of columns, the aligned enlarged fluidic channels of minimum width EW₁ and the aligned enlarged fluidic channels of minimum width EW₂ being disposed within the receptacle to promote a cross-movement of fluid medium through the receptacle in addition to a main axial flow of fluid medium through the receptacle from the inlet end to the outlet end thereof.

11. The apparatus of claim 1, wherein the microstructures comprise at least one of cells, viruses, bacteria, macromolecules, or minute particles.

12. The apparatus of claim 1, wherein the receptacle is fabricated at least partially of a transparent polymer, and wherein fluid medium flow through the receptacle is at least one of pressure driven or electrophoretically driven.

13. The apparatus of claim 12, wherein fluid medium flow through the receptacle from the inlet to the outlet thereof comprises a main axial flow, and wherein the apparatus further comprises at least one cross-flow inlet and at least one cross-flow outlet for establishing at least one cross-flow of fluid through the receptacle in a direction which intersects a main axial flow through the receptacle, the at least one cross-flow facilitating removal of sorted microstructures from the receptacle, and wherein the main axial flow is at least one of pressure or electrophoresis driven, and the at least one cross-flow is at least one of pressure or electrophoresis driven.

14. A method of sorting microstructures in a fluid medium, the method comprising:
providing a receptacle having N regions of columns positioned in the receptacle between an inlet and an outlet thereof, wherein N≧2 and fluid medium introduced into the receptacle through the inlet passes sequentially through the N regions of columns before exiting through the outlet, and wherein each region i (i=1 . . . N) of columns of the N regions of columns comprises at least one row of columns spaced to define multiple fluidic channels of a respective minimum width Wᵢ, and wherein the minimum widths Wᵢ of the multiple fluidic channels of the at least one row of each region of columns (1 . . . N) decrease in size in the receptacle between the N regions of columns from the inlet to the outlet thereof; and
employing the receptacle to sort microstructures in a fluid medium by introducing the fluid medium with the microstructures therein into the receptacle through the inlet and allowing the fluid medium to pass through the N regions of columns before exiting through the outlet, wherein differently sized microstructures separate in different regions of the receptacle dependent, in part, on physical characteristics thereof.

15. The method of claim 14, wherein the providing comprises providing the receptacle with a first region of columns and a second region of columns positioned in the receptacle between the inlet and the outlet thereof, wherein fluid medium introduced into the receptacle through the inlet passes through the first region of columns and then the second region of columns before exiting through the outlet, and wherein the first region of columns comprises at least one row of columns spaced and sized to define multiple fluidic channels of minimum width W₁ and minimum depth D₁ and the second region of columns comprises at least one row of columns spaced and sized to define multiple fluidic channels of minimum width W₂ and minimum depth D₂, wherein W₁>W₂ and D₁>D₂.

16. The method of claim 14, wherein the providing comprises providing the receptacle to comprise a first region of columns and a second region of columns positioned in the receptacle between the inlet and the outlet thereof, wherein fluid medium introduced into the receptacle through the inlet passes through the first region of columns and then the second region of columns before exiting through the outlet, and wherein the first region of columns comprises multiple rows of columns, each row having columns spaced to define multiple fluidic channels of minimum width W₁ and at least some rows of the multiple rows of the first region of columns having at least one enlarged fluidic channel of minimum width EW₁, and wherein the second region of columns comprises multiple rows of columns, each row having columns spaced to define multiple fluidic channels of minimum width W₂ and at least some rows of the multiple rows of the second region of columns each having at least one enlarged fluidic channel of minimum width EW₂, wherein EW₁>W₁ and EW₂>W₂.

17. The method of claim 16, wherein the providing comprises providing the receptacle with the enlarged fluidic channels of minimum width EW₁ and EW₂ disposed to facilitate non-axial movement of fluid medium within the receptacle in addition to axial flow of fluid medium through the receptacle from the inlet to the outlet thereof.

18. The method of claim 17, wherein the providing comprises providing the receptacle with unaligned enlarged fluidic channels of minimum width EW₁ in at least some successive rows of the multiple rows of columns of the first region of columns, and providing the receptacle with unaligned enlarged fluidic channels of minimum width EW₂ in at least some successive rows of the multiple rows of columns of the second region of columns.

19. The method of claim 17, wherein the providing comprises providing the receptacle with the outlet disposed at an outlet end of the receptacle, and the inlet at an inlet end of the receptacle, the outlet being disposed at one side of the outlet end of the receptacle, and wherein the enlarged fluidic channels of minimum width EW₁ of the first region of columns are aligned in at least some successive rows of the multiple rows of columns of the first regions of columns, and the enlarged fluidic channels of minimum width EW₂ of the second region of columns are aligned in at least some successive rows of the multiple rows of columns of the second region of columns, the aligned enlarged fluidic channels of minimum width EW₁ and the aligned enlarged fluidic channels of minimum width EW₂ being disposed within the receptacle to promote a cross-movement of fluid medium through the receptacle in addition to a main axial flow of fluid medium through the receptacle from the inlet end to the outlet end thereof.

20. The method of claim 14, further comprising facilitating fluid medium flow through the receptacle by at least one of applying a pressure differential between the inlet and outlet thereof or employing an electromotive force between the inlet and outlet.

21. The method of claim 20, wherein the providing further comprises providing the receptacle with at least one cross-flow inlet and at least one cross-flow outlet for establishing at least one cross-flow of fluid through the receptacle in a direction which intersects a main axial flow through the receptacle, the at least one cross-flow facilitating removal of sorted microstructures from the receptacle, and wherein the at least one cross-flow of fluid is facilitated by providing at least one of a pressure differential between the at least one cross-flow inlet and the at least one cross-flow outlet or an electromotive force between the at least one cross-flow inlet and the at least one cross-flow outlet.

22. The method of claim 14, further comprising implementing the method in a hand-held apparatus, wherein the providing comprises providing the receptacle as a replaceable and disposable cartridge within the hand-held apparatus.

23. The method of claim 14, wherein the providing comprises fabricating the receptacle to comprise a transparent polymer for facilitating viewing of the microstructures and fluid medium when disposed within the receptacle.

24. The method of claim 14, further comprising:
providing multiple receptacles, each having multiple regions of columns positioned in the receptacle between an inlet and an outlet thereof, wherein fluid medium introduced into each receptacle through the inlet passes sequentially through the multiple regions of columns before exiting through the outlet, and wherein each region of columns of the multiple regions of columns comprises at least one row of columns spaced to define multiple fluidic channels of minimum width Wᵢ, and wherein the minimum widths Wᵢ of the multiple fluidic channels of the at least one row of each region of columns varies between the multiple regions of columns and decrease in size in the receptacle between the regions of columns from the inlet to the outlet thereof, and wherein different receptacles have different regions of columns spaced to define at least some fluidic channels of different minimum widths Wᵢ; and
providing a shared inlet plenum coupled to an inlet of each receptacle of the multiple receptacles for receiving fluid medium having the microstructures therein for sorting, wherein the different regions of columns of the different receptacles facilitate testing of critical dimensions of one or more microstructures in the fluid medium.
